(19) United States
(12) Patent Application Publication
(10) Pub. No.: US 2023/0079923 A1
(43) Pub. Date: Mar. 16, 2023

(54) SELF-CALIBRATING SYSTEM FOR PRODUCING ELECTRICAL SIGNAL OUTPUT

(71) Applicant: International Business Machines Corporation, Armonk, NY (US)

(72) Inventors: Andrea Simonetto, Tours (FR); Tigran Tigran Tchrakian, Dublin (IE); Pritish Ranjan Parida, Cortlandt Manor, NY (US)

(21) Appl. No.: 17/472,174

(22) Filed: Sep. 10, 2021

(51) Int. Cl. A61N 1/36; G06F 30/20; G16H 50/20; G16H 20/30; G16H 40/40; G16H 40/63; G16H 10/60

(52) U.S. Cl. CPC A61N 1/36189; G06F 30/20; G16H 50/20; G16H 20/30; G16H 40/40; G16H 40/63; G16H 10/60; G16H 20/70

(57) ABSTRACT

One or more systems, computer-implemented methods and/or computer program products to facilitate a process to produce a specified electrical output are provided. A system can comprise a memory that stores computer executable components and a processor that executes the computer executable components stored in the memory. The computer executable components can comprise an inverse modeling component that can generate an entity-based model and can determine updated one or more electrostatics parameters based on feedback relative to application of an electrical stimulation therapy employing an initial one or more electrostatics parameters on the entity. In one or more embodiments, a configuration component can determine the initial one or more electrostatics parameters based on an initial physical-based model based on an ideal entity, the configuration component can generate an initial physical-based model based on an ideal entity, and/or the inverse modeling component can update the initial model to generate the entity-based model.

### BACKGROUND

One or more embodiments described herein relate to determining one or more electrostatics parameters and producing an electrical output, and more specifically to generating and recalibrating an entity-based model to determine one or more updated electrostatics parameters for producing a specified electrical output, such as an electrical signal output.

Today, electrical stimulation therapy, such as including neuromodulation, can be performed based on one or more idealized models. Using mathematical results, determined relative to the idealized models, and often stored in one or more lookup tables, electrical output can be adjusted employing the lookup tables. An actual electrical output produced typically does not initially match a specified electrical output requested. One or more configurations and/or parameters can be adjusted to produce an updated actual electrical output based on the idealized model. It will be appreciated that the updated actual electrical output can provide a specified result, although the actual electrical output can be indirectly related to the idealized model.

### SUMMARY

The following presents a summary to provide a basic understanding of one or more embodiments described herein. This summary is not intended to identify key or critical elements, or to delineate any scope of the particular embodiments and/or any scope of the claims. The sole purpose of the summary is to present concepts in a simplified form as a prelude to the more detailed description that is presented later. In one or more embodiments described herein, devices, systems, computer-implemented methods, apparatuses and/or computer program products are described that can facilitate a process to produce a specified electrical output, such as an electrical signal output.

According to an embodiment, a system can comprise a processor that executes computer executable components stored in memory, where the computer executable components comprise an inverse modeling component that generates an entity-based model and determines updated one or more electrostatics parameters based on feedback relative to application of an electrical stimulation therapy employing an initial one or more electrostatics parameters on the entity.

According to another embodiment, a computer-implemented method can comprise generating, by a system operatively coupled to a processor, an entity-based model. The computer-implemented method also can comprise determining, by the system, updated one or more electrostatics parameters based on feedback relative to application of an electrical stimulation therapy employing an initial one or more electrostatics parameters on the entity.

According to yet another embodiment, a computer program product can facilitate a process for producing a specified electrical output, the computer program product comprising a computer readable storage medium having program instructions embodied therewith, the program instructions executable by a processor to cause the processor to, generate, by the processor, an entity-based model, and determine, by the processor, updated one or more electrostatics parameters based on feedback relative to application of an electrical stimulation therapy employing an initial one or more electrostatics parameters on the entity.

### DESCRIPTION OF THE DRAWINGS

FIG. 1 illustrates a block diagram of an example, non-limiting system that can facilitate a process to produce a specified electrical output in accordance with one or more embodiments described herein.

FIG. 2 illustrates another block diagram of an example, non-limiting system that can facilitate a process to produce a specified electrical output in accordance with one or more embodiments described herein.

FIG. 3 illustrates a flow diagram of a process facilitated by the non-limiting system of FIG. 2, in accordance with one or more embodiments described herein.

FIG. 4A illustrates a representation of a spine of an entity acted upon by an electrical output that can be produced by the non-limiting system of FIG. 2, in accordance with one or more embodiments described herein.

FIG. 4B illustrates a representation of a portion of a spine of an entity acted upon by an electrical output that can be produced by the non-limiting system of FIG. 2, in accordance with one or more embodiments described herein.

FIG. 5 illustrates another flow diagram of a process facilitated relative to the spine of an entity as illustrated at FIGS. 4A and 4B, in accordance with one or more embodiments described herein.

FIG. 6 illustrates yet another block diagram of an example, non-limiting system that can facilitate a process to produce a specified electrical output in accordance with one or more embodiments described herein.

FIG. 7 illustrates a flow diagram of an example, non-limiting computer-implemented method that can facilitate a process to produce a specified electrical output, in accordance with one or more embodiments described herein.

FIG. 8 illustrates a continuation of the flow diagram of FIG. 7, of an example, non-limiting computer-implemented method that can facilitate a process to produce a specified electrical output, in accordance with one or more embodiments described herein.

FIG. 9 illustrates a block diagram of an example, non-limiting operating environment in which one or more embodiments described herein can be facilitated.

FIG. 10 illustrates a block diagram of an example, non-limiting cloud computing environment in accordance with one or more embodiments described herein.

FIG. 11 illustrates a block diagram of a plurality of example, non-limiting abstraction model layers, in accordance with one or more embodiments described herein.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in this Detailed Description section.

Given existing approaches to determine one or more electrostatics parameters based on an idealized model, such as an idealized physical-based model, a system, method, process and/or computer-program product is specified that can reduce the amount of educated guesswork, logic-based interpolation and/or iterations employed to determine the one or more electrostatics parameters for producing a specified electrical output, such as an electrical signal output. That is, it is often the case that electrical output is delivered via placement of output targets, e.g., electrodes, leads and/or other devices, that can be not directly and/or fully visible to an administrating entity. As used herein, the term “entity” can refer to a machine, device, smart device, component, hardware, software and/or human.

While use of vision devices, software and/or machines can be employed to indirectly view placement of the output targets, such output targets can migrate and exact location of the output targets, relative to a model upon which initial electrostatics parameters were determined, can be different. That is, although employing an idealized model and associated lookup table, such initial constructs (e.g., idealized model and associated lookup table) often simply do not exactly correspond to the actual entity at which the electrical output is being directed. Further, while iterations of updated electrostatic parameters can be performed to finally achieve an actual electrical output that matches an updated specified electrical output, such existing technique can employ unwanted amounts of time, energy and/or computing power. Further, such updated specified electrical output can be different from an initial specified electrical output, thus causing issue and/or concern relative to reconstructing the respective electrical stimulation therapy parameters.

One or more embodiments of one or more systems, computer-implemented methods and/or computer-program products described herein can address one or more of these deficiencies. The one or more embodiments described here can be employed to determine updated one or more electrostatics parameters for being used to produce an updated electrical output. The electrical output produced can be more quickly and more efficiently matched to a specified electrical output than using existing techniques. This is because the one or more embodiments described herein can facilitate generation, recalibration and/or updating of a computer-generated model of the entity. Such entity-based model can be generated based upon entity feedback, which can be obtained directly and/or indirectly from and/or relative to the entity upon which the electrical output(s) is being employed (also referred to herein as a subject entity). Indeed, alternative to employing a single, idealized model and associated lookup table to determine one or more parameters from the lookup table that do not ideally correspond to the actual electrical output produced on a subject entity, the one or more embodiments described herein can employ inverse modelling to facilitate better, closer and/or more efficient determination of one or more electrostatics parameters based upon an entity-based model. Indeed, by employing inverse modeling, although only patterns and output often can be observed, and not the exact process (e.g., due to the output targets being embedded, buried and/or otherwise within the subject entity) the unobserved process can be inferred, and entity feedback employed in an inverse manner. The model upon which electrostatics parameters are based, e.g., utilized in the determination of the electrostatics parameters, can be updated, recalibrated and or generated to more closely align to the subject entity, such as through one or more iterations.

In this way, the determined one or more parameters can ideally correspond both to an ideal electrical output relative to the entity-based model, upon which the one or more parameters are based, and to an actual electrical output at the subject entity. Because the model-based electrical output and actual electrical output can match and/or at least more closely match, than as facilitated by existing techniques, the entity-based model can be employed for more than one electrical output, type of electrical output, output session and/or future use. Overhead, such as time, power and/or computer usage, can be reduced for additional electrical outputs. Further, where one or more embodiments described herein can be employed relative to an entity that is live, subject entity discomfort can be reduced, and specified electrical output can be produced more quickly and/or efficiently. In one or more cases, such output can modulate signals traveling from an extremity through the spinal cord to the brain, such as to improve extremity movement, such as walking. As such, generating an entity-based model can directly aid the subject entity.

One or more embodiments are now described with reference to the drawings, where like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

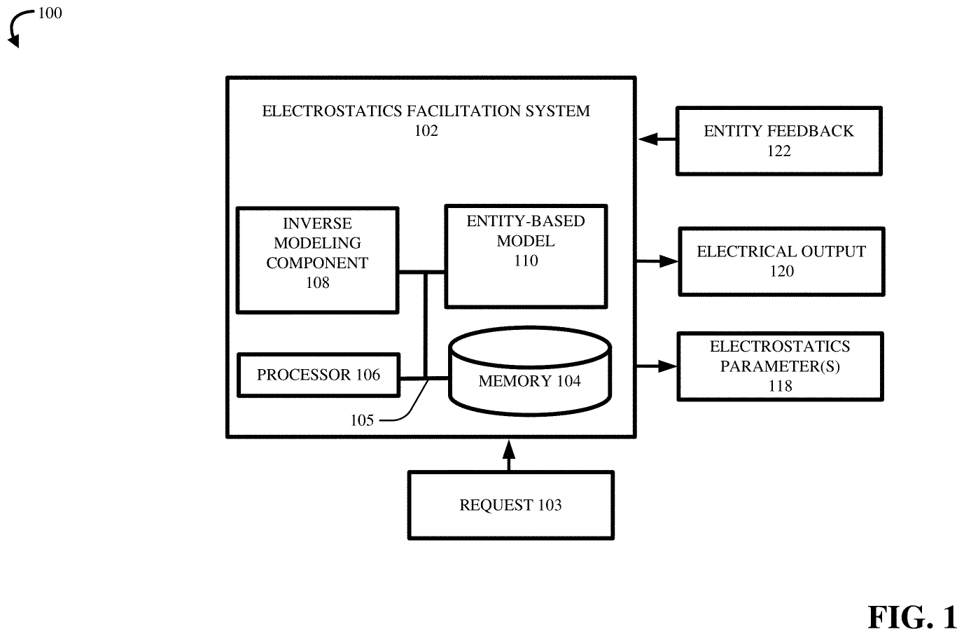

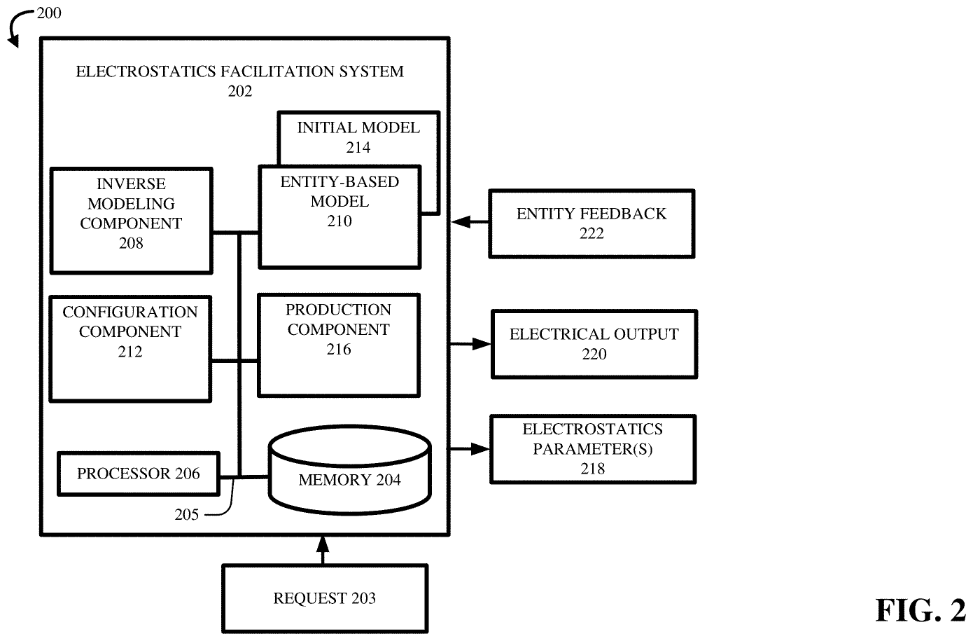

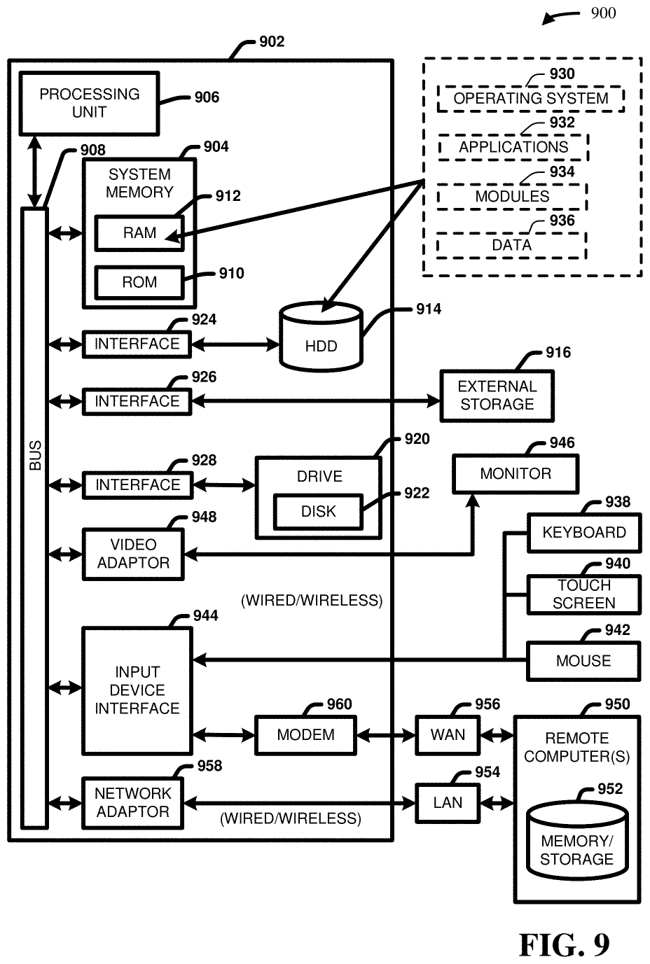

Further, it will be appreciated that the embodiments depicted in one or more figures described herein are for illustration only, and as such, the architecture of embodiments is not limited to the systems, devices and/or components depicted therein, nor to any particular order, connection and/or coupling of systems, devices and/or components depicted therein. For example, in one or more embodiments, the non-limiting systems 100, 200 and/or 600 as illustrated at FIGS. 1, 2 and/or 6, and/or systems thereof, can further comprise one or more computer and/or computing-based elements described herein with reference to an operating environment, such as the operating environment 900 illustrated at FIG. 9. In one or more described embodiments, computer and/or computing-based elements can be used in connection with implementing one or more of the systems, devices, components and/or computer-implemented operations shown and/or described in connection with FIGS. 1, 2 and/or 6 and/or with other figures described herein.

Turning first generally to FIG. 1, illustrated is a block diagram of an example, non-limiting system 100 that can facilitate a process for producing an electrical output to a subject entity, also referred to herein as an entity, in accordance with one or more embodiments described herein. The non-limiting system 100 can be employed for producing electrical outputs such as various electrostatics fields, electrostatics waveforms, sub-perception stimulation, neuromodulation stimulations, muscle stimulations, brain stimulation and/or other related uses. All electrical outputs and/or types thereof are envisioned. Indeed, the non-limiting system 100 can be employed for application of electrical stimulation therapy to one or more varying entities, which can include inanimate, live and/or deceased entities. While referring here to one or more processes, facilitations and/or uses of the non-limiting system 100, it will be appreciated that description provided herein, both above and below, also can be relevant to one or more other non-limiting systems described herein, such as the non-limiting systems 200 and 600, to be described below in detail.

As illustrated at FIG. 1, the non-limiting system 100 can comprise an electrostatics facilitation system 102. Electrostatics facilitation system 102 can comprise one or more components, such as a memory 104, processor 106, bus 105, inverse modeling component 108 and/or entity-based model 110. Generally, electrostatics facilitation system 102 can facilitate generation of one or more entity-based models and/or determination of one or more electrostatics parameters relative to an request 103. The inverse modeling component 108 and entity-based model 110 of the electrostatics facilitation system 102 can participate in an exemplary production and/or determination, to be discussed below in detail.

One or more aspects of a component can be employed separately and/or in combination, such as employing one or more of a memory or a processor of a system that includes the component to thereby facilitate determination of one or more electrostatics parameters. The inverse modeling component 108 can employ the entity-based model 110, processor 106 and/or the memory 104. Additionally and/or alternatively, the processor 106 can execute one or more program instructions to cause the processor to perform one or more operations by the inverse modeling component 108 and/or employing the entity-based model 110.

Turning now to one or more operations of the electrostatics facilitation system 102, the inverse modeling component 108 and/or the processor 106 can receive the request 103. The request 103 can be received and/or retrieved by any suitable means. It will be appreciated that the request 103 can be directed to production of a particular (e.g., specified) electrical output relative to a particular entity (e.g., subject entity). The request 103 can be provided in any suitable format, such as a text format, binary format and/or another suitable format. In one or more embodiments, the request 103 can be received by the electrostatics facilitation system 102, such as by a component and/or aspect of the electrostatics facilitation system 102 other than the inverse modeling component 108, such as the memory 104, a mail component and/or a download component.

The request 103 can comprise a specified electrical output, which can include a location of output, frequency of output, one or more varying levels of output, type of output, timing of output and/or any other suitable aspect related to the specified electrical output. Output type requested can include one or more signals, wavelengths and/or energy types. For example, in one or more embodiments, current amplitude can be about 0 mA to about 50 mA, pulse/signal rate/frequency can be about 1 Hz to about 50 kHz, and/or signal-/pulse-width can be about 1 us to about 1000 us. Indeed, current can be distributed across a plurality of output targets, such as in the range of about 32 output targets to about 100 output targets. It will be appreciated that the request 103 can be a secondary or updated request to the electrostatics facilitation system 102, such as after an initial electrical output 120 employing an initial one or more electrostatics parameters 118 on an entity.

Entity feedback 122, relative to application of an electrical stimulation therapy employing the initial one or more electrostatics parameters 118 on the entity, also can be provided via any aforementioned suitable means and/or method to the electrostatics facilitation system 102. The entity feedback 122 can include one or more differentials between the specified electrical output requested and the actual electrical output 120 produced. The one or more differentials can include difference in location, strength, frequency and/or other suitable aspect relative to the actual electrical output.

Employing the entity feedback 122 and request 103, the inverse modeling component 108 can generate an entity-based model 110 and determine updated one or more electrostatics parameters 118. That is, employing the one or more observed patterns from a produced electrical output, e.g., employing the results as input, the inverse modeling component 108 can revise the electrical stimulation therapy process through one or more iterations. The inverse modeling component 108 can, employing the one or more differentials, utilize artificial intelligence (AI) model, machine learning (ML) model and/or like model to generate a more accurate model to the subject entity than an idealized physical model. That is, the inverse modeling component 108 can generate an entity-based model 110. This generation can be of a new entity-based model 110 or of an update or recalibration of a previous model, such as an initial idealized model.

The models can be physical-based, meaning that the models can be based on actual physical aspects in the real world. Such models can employ one or more 3D parameters, although data can be stored employing one dimensional and/or two dimensional vectors and/or other data aspects, such as via one or more lookup tables.

Employing the generated entity-based model 110, the inverse modeling component 108 can generate, update and/or revise a previous lookup table. The lookup table can include one or more results, such as output target configurations, locations, output strengths, output types, output frequencies and/or other aspects. The lookup table results can be generated employing one or more methods understood by one having ordinary skill in the art. For example, various mathematics and/or computer-related models can be employed, such as depending on the entity receiving the therapy, and/or on the location of the entity receiving therapy. For example, relative to spinal electrical stimulation therapy, a Poisson model of the spine can be employed to provide a set of results to then be stored in a respective lookup table.

Based on the lookup table results, and on the specified electrical output, the inverse modeling component 108 can generate one or more updated electrostatics parameters 118. The one or more updated electrostatics parameters can be employed, such as by the electrostatics facilitation system 102 or other system, to produce an electrical output 120 that is more closely aligned to the specified electrical output of the request 103. Further, in view of the inverse modeling employed, an initial model can be updated and/or a new entity-based model 110 generated. The entity-based model 110 generated can be employed to generate lookup table results, and thus one or more electrostatics parameters, for achieving both the specified electrical output and one or more other electrical outputs relative to the subject entity. For example, based on the differentials of the entity feedback 122, differences between a previous (e.g., ideal) model and an entity-based model 110 can be construed for various locations of the subject entity, including locations not relevant or directly at a location of the specified electrical output. This is in opposition to maintaining only an initial idealized model and lookup table and providing one or more updated parameters for achieving the specified electrical output based on lookup table results non-coordinated to an actual electrical output, as with existing technologies. Indeed, as will be appreciated, the inverse modeling component 108 can generate one or more updated electrostatics parameters 118 that can be coordinated with both the specified electrical output and the proposed model-based electrical output.

Turning next to FIG. 2, the figure illustrates a diagram of an example, non-limiting system 200 that can facilitate a process to produce a specified electrical output. Repetitive description of like elements and/or processes employed in respective embodiments is omitted for sake of brevity. As indicated previously, description relative to an embodiment of FIG. 1 can be applicable to an embodiment of FIG. 2. Likewise, description relative to an embodiment of FIG. 2 can be applicable to an embodiment of FIG. 1.

As illustrated, the non-limiting system 200 can include an electrostatics facilitation system 202. The electrostatics facilitation system 202, as illustrated, can comprise any suitable type of component, machine, device, facility, apparatus and/or instrument that comprises a processor and/or can be capable of effective and/or operative communication with a wired and/or wireless network. All such embodiments are envisioned. For example, electrostatics facilitation system 202 can comprise a server device, computing device, general-purpose computer, special-purpose computer, quantum computing device (e.g., a quantum computer), tablet computing device, handheld device, server class computing machine and/or database, laptop computer, notebook computer, desktop computer, cell phone, smart phone, consumer appliance and/or instrumentation, industrial and/or commercial device, digital assistant, multimedia Internet enabled phone, multimedia players and/or another type of device and/or computing device. Likewise, the electrostatics facilitation system 202 can be disposed and/or run at any suitable device, such as, but not limited to a server device, computing device, general-purpose computer, special-purpose computer, quantum computing device (e.g., a quantum computer), tablet computing device, handheld device, server class computing machine and/or database, laptop computer, notebook computer, desktop computer, cell phone, smart phone, consumer appliance and/or instrumentation, industrial and/or commercial device, digital assistant, multimedia Internet enabled phone, multimedia players and/or another type of device and/or computing device.

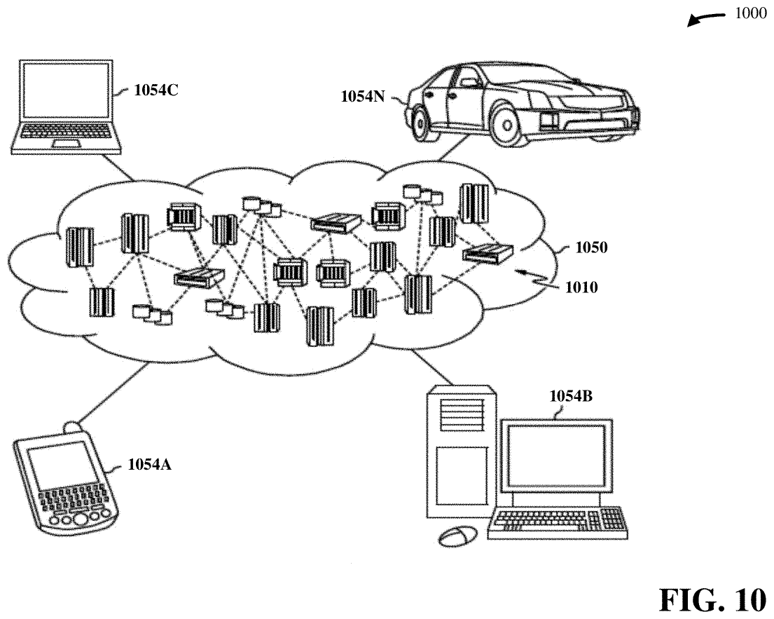

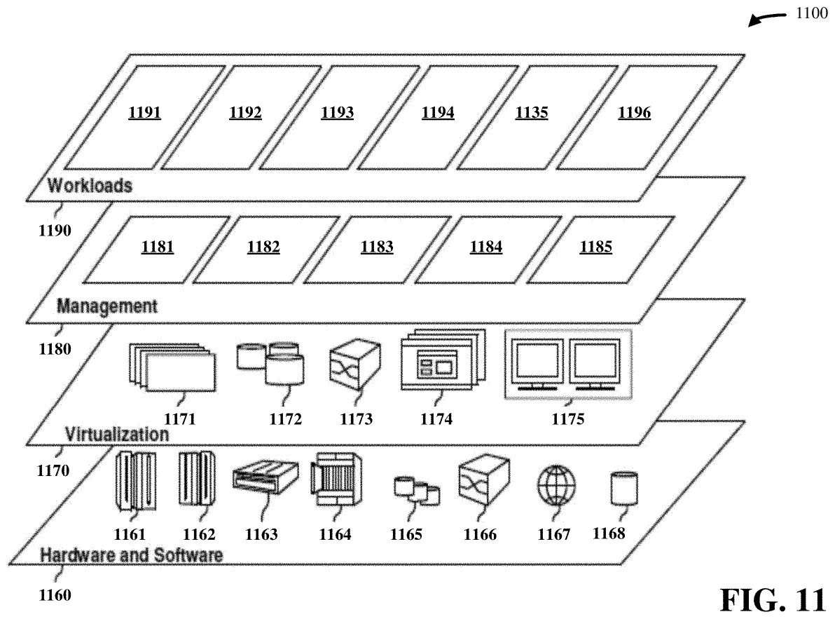

The electrostatics facilitation system 202 can be associated with, such as accessible via, a cloud computing environment. For example, the electrostatics facilitation system 202 can be associated with a cloud computing environment 1050 described below with reference to FIG. 10 and/or with one or more functional abstraction layers described below with reference to FIG. 11 (e.g., hardware and software layer 1160, virtualization layer 1170, management layer 1180 and/or workloads layer 1190).

It will be appreciated that operation of the non-limiting system 200 and/or of the electrostatics facilitation system 202 is not limited to execution of a single request at a time. Rather, operation of the non-limiting system 200 and/or of the electrostatics facilitation system 202 can be scalable. For example, the non-limiting system 200 and/or the electrostatics facilitation system 202 can facilitate determination of one or more parameters, production of one or more electrical outputs and/or generation of one or more entity-based models relative to one or more entities in parallel with execution of one or more determinations, productions and/or generations. In one or more embodiments, one or more components of the non-limiting system 200 and/or of the electrostatics facilitation system 202 can be employed to execute at the same time two or more determinations, productions and/or generations including the one or more components.

The electrostatics facilitation system 202 can comprise a plurality of components. The components can include a memory 204, processor 206, bus 205, inverse modeling component 208, entity-based model 210, configuration component 212, initial model 214 and/or production component 216. One or more of these components can be employed to address a request 203 and to generate one or more electrostatics parameters 218 and/or one or more electrical outputs 220, such as based on one or more entity feedbacks 222. Like the electrostatics facilitation system 102, the electrostatics facilitation system 202 can be operated to generate an entity-based model 210 and to provide the one or more updated electrostatics parameters 218, such as based on and/or employing the entity-based model 210. It also will be appreciated that the electrostatics facilitation system 202 can execute the electrical output 220, such as via the production component 216.

The request 203 can be received and/or retrieved by any suitable means. The component, system and/or other aspect receiving (e.g., initially and/or indirectly) the request 203 can employ any one or more aspects of an operating environment, such as the operating environment 900 (FIG. 9), to receive and/or retrieve the request 203. By way of a non-limiting example, the request 103 can be uploaded from the HDD 914, received from the memory/storage 952 via the WAN 956 and/or downloaded via the WAN 956 from a node, such as a cloud computing node 1010 of a cloud computing environment 1050 (FIG. 10).

One or more communications between one or more components of the non-limiting system 200, and/or between an entity providing the request 203 and the non-limiting system 200, can be facilitated by wired and/or wireless means including, but not limited to, employing a cellular network, a wide area network (WAN) (e.g., the Internet), and/or a local area network (LAN). Suitable wired or wireless technologies for facilitating the communications can include, without being limited to, wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Zigbee and other 802.XX wireless technologies and/or legacy telecommunication technologies, BLUETOOTH®, Session Initiation Protocol (SIP), ZIGBEE®, RF4CE protocol, WirelessHART protocol, 6LoWPAN (Ipv6 over Low power Wireless Area Networks), Z-Wave, an ANT, an ultra-wideband (UWB) standard protocol and/or other proprietary and/or non-proprietary communication protocols.

Discussion now turns to the processor 206, memory 204 and bus 205 of the electrostatics facilitation system 202.

For example, in one or more embodiments, electrostatics facilitation system 202 can comprise a processor 206 (e.g., computer processing unit, microprocessor, classical processor, quantum processor and/or like processor). In one or more embodiments, a component associated with electrostatics facilitation system 202, as described herein with or without reference to the one or more figures of the one or more embodiments, can comprise one or more computer and/or machine readable, writable and/or executable components and/or instructions that can be executed by processor 206 to facilitate performance of one or more processes defined by such component(s) and/or instruction(s). In one or more embodiments, the processor 206 can comprise the inverse modeling component 208, configuration component 212 and/or production component 216.

In one or more embodiments, the electrostatics facilitation system 202 can comprise a computer-readable memory 204 that can be operably connected to the processor 206. The memory 204 can store computer-executable instructions that, upon execution by the processor 206, can cause the processor 206 and/or one or more other components of the electrostatics facilitation system 202 (e.g., inverse modeling component 208, configuration component 212 and/or production component 216) to perform one or more actions. In one or more embodiments, the memory 204 can store computer-executable components (e.g., inverse modeling component 208, configuration component 212 and/or production component 216).

Electrostatics facilitation system 202 and/or a component thereof as described herein, can be communicatively, electrically, operatively, optically and/or otherwise coupled to one another via a bus 205 to perform functions of non-limiting system 200, electrostatics facilitation system 202 and/or one or more components thereof and/or coupled therewith. Bus 205 can comprise one or more of a memory bus, memory controller, peripheral bus, external bus, local bus, quantum bus and/or another type of bus that can employ one or more bus architectures. One or more of these examples of bus 205 can be employed to implement one or more embodiments described herein.

In one or more embodiments, electrostatics facilitation system 202 can be coupled (e.g., communicatively, electrically, operatively, optically and/or like function) to one or more external systems (e.g., a non-illustrated electrical output production system, one or more output targets, an output target controller and/or the like), sources and/or devices (e.g., classical and/or quantum computing devices, communication devices and/or like devices), such as via a network. In one or more embodiments, one or more of the components of the non-limiting system 200 can reside in the cloud, and/or can reside locally in a local computing environment (e.g., at a specified location(s)).

In addition to the processor 206 and/or memory 204 described above, electrostatics facilitation system 202 can comprise one or more computer and/or machine readable, writable and/or executable components and/or instructions that, when executed by processor 206, can facilitate performance of one or more operations defined by such component(s) and/or instruction(s).

Turning now to the configuration component 212, the configuration component 212 can determine an initial one or more electrostatics parameters 218, such as based on an initial physical-based model 214 based on an ideal entity (e.g., an idealized model). The configuration component 212 can utilize artificial intelligence (AI) model, machine learning (ML) model and/or like model to generate the initial model 214. Alternatively, the initial model 214 can be provided to the configuration component 212. Employing the initial model 214, the configuration component 212 can generate, update and/or revise a previous lookup table. Alternatively, the lookup table can be provided to the configuration component 212. The lookup table can include one or more results, such as output target configurations, locations, output strengths, output types, output frequencies and/or other aspects. The lookup table results can be generated employing one or more methods understood by one having ordinary skill in the art. For example, various mathematics and/or computer-related models can be employed, such as depending on the entity receiving the therapy, and/or on the location of the entity receiving therapy. For example, relative to spinal electrical stimulation therapy, a Poisson model of the spine can be employed to provide a set of results to then be stored in a respective lookup table.

Turning next to the inverse modeling component 208, function can parallel that described above with respect to the inverse modeling component 108. That is, employing entity feedback 222 and the request 203, the inverse modeling component 208 can generate an entity-based model 210 and determine updated one or more electrostatics parameters 218. That is, employing the one or more observed patterns from a an initial produced electrical output 220, e.g., employing the results as input, the inverse modeling component 208 can revise the electrical stimulation therapy process being performed on a subject entity through one or more iterations.

Entity feedback 222, relative to application of an initial electrical stimulation therapy employing the initial one or more electrostatics parameters 218 on the entity, can be provided via any aforementioned suitable means and/or method to the electrostatics facilitation system 202. For example, an administrating entity can visually, electronically and/or otherwise determine one or more feedbacks 222; the subject entity can directly provide one or more feedbacks 222 such as orally, manually or otherwise; and/or a computer entity can take and/or receive one or more signals, results, measurements and/or other readings from the subject entity. The entity feedback 222 can include one or more differentials between the specified electrical output requested and the actual electrical output 220 produced. The one or more differentials can include difference in location, strength, frequency, signal-/pulse-width, pulse duration, electrical field size (e.g., electrical field coverage) and/or other suitable aspect relative to the actual electrical output.

Employing the entity feedback 222 and request 203, the inverse modeling component 208 can generate the entity-based model 210 and determine updated one or more electrostatics parameters 218. That is, employing the one or more observed patterns from the produced electrical output, e.g., employing the results as input, the inverse modeling component 208 can revise the electrical stimulation therapy process through one or more iterations. The inverse modeling component 208 can, employing the one or more differentials, utilize artificial intelligence (AI) model, machine learning (ML) model and/or like model to generate a more accurate model to the subject entity than an idealized physical model. That is, the inverse modeling component 208 can generate an entity-based model 210. This generation can be of a new entity-based model 210 or of an update or recalibration of a previous model, such as of the initial model 214. Put another way, the generation (e.g., update, recalibration and/or the like) can be performed between a determination of the initial one or more electrostatics parameters 218 and the determination of the updated one or more electrostatics parameters 218.

With respect to the updated one or more electrostatics parameters 218, employing the generated entity-based model 210, the inverse modeling component 208 can generate, update and/or revise a previous lookup table. The lookup table can include one or more results, such as output target configurations, locations, output strengths, output types, output frequencies and/or other aspects. The lookup table results can be generated employing one or more methods understood by one having ordinary skill in the art. For example, various mathematics and/or computer-related models can be employed, such as depending on the entity receiving the therapy, and/or on the location of the entity receiving therapy. For example, relative to spinal electrical stimulation therapy, a Poisson model of the spine can be employed to provide a set of results to then be stored in a respective lookup table.

Based on the lookup table results, and on the specified electrical output, the inverse modeling component 208 can generate one or more updated electrostatics parameters 218. The one or more updated electrostatics parameters can be employed, such as by the electrostatics facilitation system 202 or other system, to produce an electrical output 220 that is more closely aligned to the specified electrical output of the request 203. Further, in view of the inverse modeling employed, an initial model can be updated and/or a new entity-based model 210 generated. The entity-based model 210 generated can be employed to generate lookup table results, and thus one or more electrostatics parameters, for achieving both the specified electrical output and one or more other electrical outputs relative to the subject entity. For example, based on the differentials of the entity feedback 222, differences between the initial model 214 and the entity-based model 210 can be construed for various locations of the subject entity, including locations not relevant or directly at a location of the specified electrical output. Indeed, as will be appreciated, the inverse modeling component 208 can generate one or more updated electrostatics parameters 218 that can be coordinated with both the specified electrical output and the proposed model-based electrical output.

Referring now to the production component 216, the updated electrical output 220 can be produced. As indicated above, the updated electrical output 220 can be based on one or more updated electrostatics parameters 218. The production component 216 can produce the updated electrical output 220 for being applied to the subject entity and/or can apply the updated electrical output 220 to the subject entity. For example, in one or more embodiments, the production component 216 can produce an electrical signal output such as a current pulse of about 1 mA at a rate of about 50 Hz utilizing all or a subset of output targets, where the distribution of the current pulse across the output targets can be determined using the one or more updated electrostatics parameters 218.

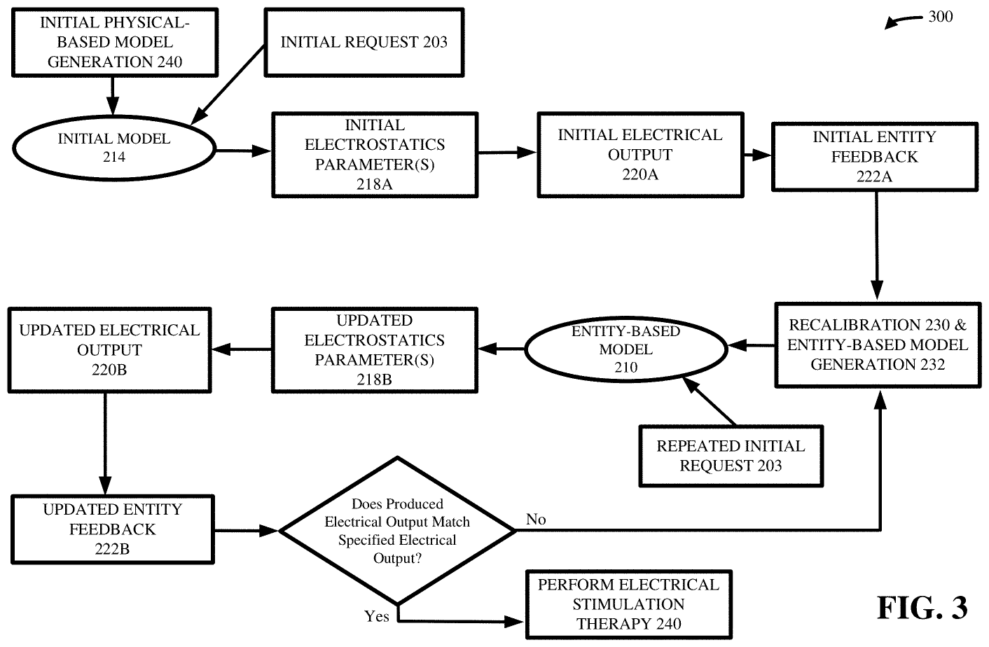

Turning now briefly to FIG. 3, the figure illustrates a diagram of an example, non-limiting process flow diagram 300 relative to the non-limiting system 200 and electrostatics facilitation system 202. Repetitive description of like elements and/or processes employed in respective embodiments is omitted for sake of brevity.

Referring to a start of an iteration process at the diagram 300 (e.g., upper left corner), an initial model 214 can be generated via an initial physical-based model generation 240. This can be facilitated external to the electrostatics facilitation system 202 and/or by the configuration component 212. An initial request 203 can be sent and employed at the initial model 214, such as being sent by an administrating entity that is administering an electrical stimulation therapy at or to a subject entity. One or more initial electrostatics parameters 218A and initial electrical outputs 220A can be determined and/or generated employing the initial model 214. The configuration component 212 can be employed to determine the one or more initial electrostatics parameters 218A, and the production component 216, for example, can be employed to produce and/or apply the initial electrical output 220A. Initial entity feedback 222A can be obtained and sent to the electrostatics facilitation system 202.

Recalibration 230 and/or entity-based model generation 232 can be performed by the inverse modeling component 208. As a result, the entity-based model 210 can be generated. A repeated initial request 203 can be sent and employed at the initial model 214, such as being sent by an administrating entity that is administering an electrical stimulation therapy at or to a subject entity. That is, rather than a secondary or additional request including a modified specified electrical output (e.g., due to differences between the initial or entity-based models 214, 210 and the actual subject entity) the same and initial specified electrical output can be repeated.

One or more updated electrostatics parameters 218B and updated electrical outputs 220B can be determined and/or generated employing the entity-based model 210. The inverse modeling component 208 can be employed to determine the one or more initial electrostatics parameters 218B, and the production component 216, for example, can be employed to produce and/or apply the initial electrical output 220B. Updated entity feedback 222B can be obtained and sent to the electrostatics facilitation system 202. Where the electrical output matches the specified electrical output and/or provides the specified effect, electrical stimulation therapy 240 can be performed on the subject entity. That is, further iterations of electrostatics parameter determination will not be performed. Alternatively, where the electrical output does not match the specified electrical output and/or provide the specified effect, one or more additional iterations of electrostatics parameter determination can be performed, continuing to employ the entity feedback 222B results as input for the inverse modeling executed by the inverse modeling component 208.

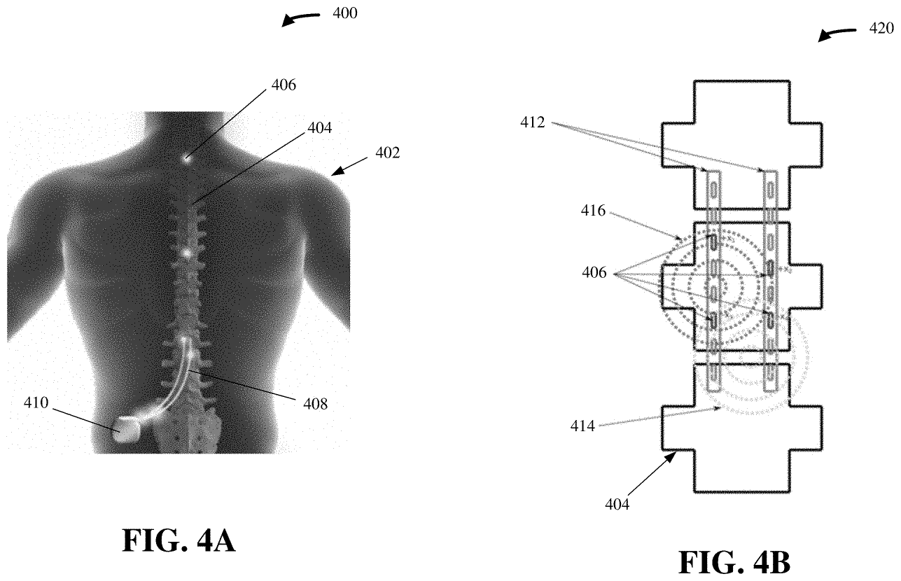

Turning now to FIGS. 4A and 4B, portions of a subject entity are illustrated as an example subject entity. As described above, the subject entity can be a live or deceased entity, such as a human entity. Again, it will be appreciated that alternative subject entities can be non-human, computer-based, organic, non-organic and/or inanimate.

The diagram 400 illustrates a portion of a subject entity 402 including the spine 404. One or more output targets 406, such as electrodes, are placed and/or located on and/or adjacent to particular locations of the spine 404. One or more leads 408 can couple the output targets 406 to a control device 410, such as internal and/or external to the subject entity 402. It will be appreciated that the control device 410 can receive and/or apply an electrical output to the spine 404 via the one or more output targets 406.

Turning next to FIG. 4B, a diagram 420 provides a different and diagrammatic view of the spine 404. One or more electrode strips 412 can be disposed on and/or adjacent to the spine 404. The electrode strips 412 each can include one or more output targets 406, e.g., electrodes. Relative to the output targets 406, a specified electrical output 414 is shown spaced from an actual electrical output 416 produced. That is, employing a respective request facilitation system and/or non-limiting system, inverse modeling can be employed to generate an entity-based model that can be employed to determine updated one or more electrical outputs for use in producing the one or more actual electrical outputs 416 until matched to and/or producing a specified effect of the illustrated specified electrical output 414.

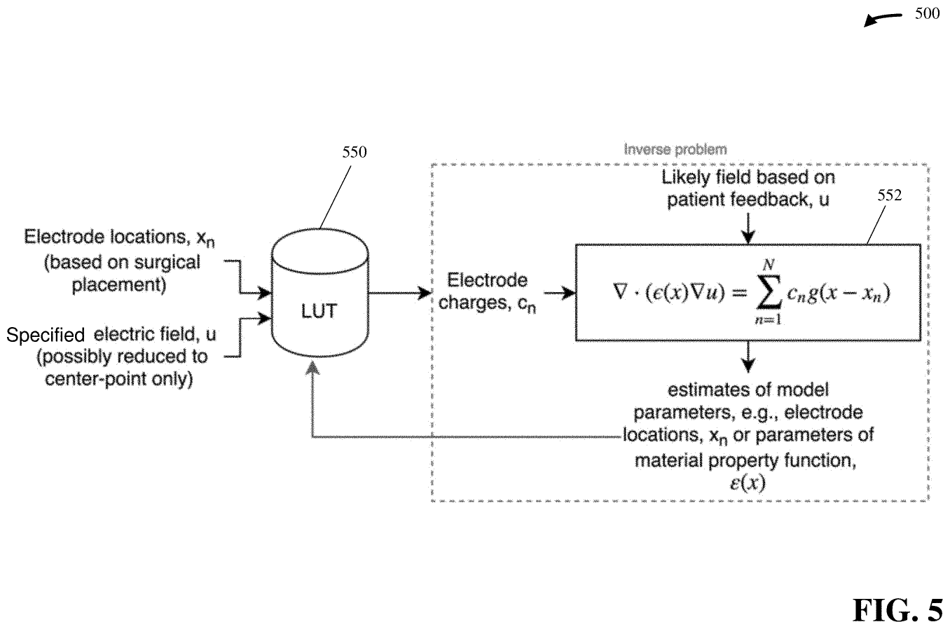

Turning now to FIG. 5, a diagram 500 is illustrated regarding generation and/or determination of one or more electrostatics parameters relative to the subject entity 402 of FIGS. 4A and 4B. That is, as shown, a lookup table 550 can be generated. The lookup table 550 can be based at least partially on output target 406 locations, such as electrode locations (xₙ), such as based on surgical placement. It will be appreciated that exact placement of the electrodes can vary due to inner-body migration and/or to how the surgical location is initially reported. The lookup table 550 also can be based at least partially on the request, which can include a request for a specified electrical output, such as a specified electric field (u). In one or more embodiments, the specified electric field can be reduced to a center-point only. Employing the lookup table 550, a respective inverse modeling component can determine one or more electrical outputs, such as electrode charges (cₙ). The electrode charges can be determined based upon use of a respective mathematical model, such as the Poisson model of the spine 552, to fill the lookup table 550. That is, for each field input (u), the lookup table 550 can be employed to set a new electrode configuration of electrode charges (cₙ).

For example, in one embodiment, a Poisson model of the spine can be expressed as Equation 1. Variable u is the scalar field, known as the electric potential field. The vector x represents 3-dimensional space, such as x=(x₁, x₂, x₃). The term, g(x−xₙ) represents the shape of the source term positioned at point xₙ. Each of the N source terms describes a charge due to an electrode at xₙ, and can be represented by a smooth function that converges to the delta function centered at xₙ. The term ε(x) represents the material properties of the spine and surrounding tissues, such as their permittivity. That is, the terms ε(x) and xₙ represent examples of electrostatics parameters employed by the respective electrostatics facilitation system.

∇*(ε(x)∇(u)=Σₙ₌₁^{N}cₙg(x−xₙ) Equation 1:

The respective electrostatics problem can be solved employing multiple sources, such as multiple electrode configurations to obtain an electric potential field corresponding to each electrode configuration. The results can be stored in a lookup table, such as the lookup table 550 and can be later used by a respective inverse modeling component to produce one or more electrode charge distributions corresponding to a specified potential field.

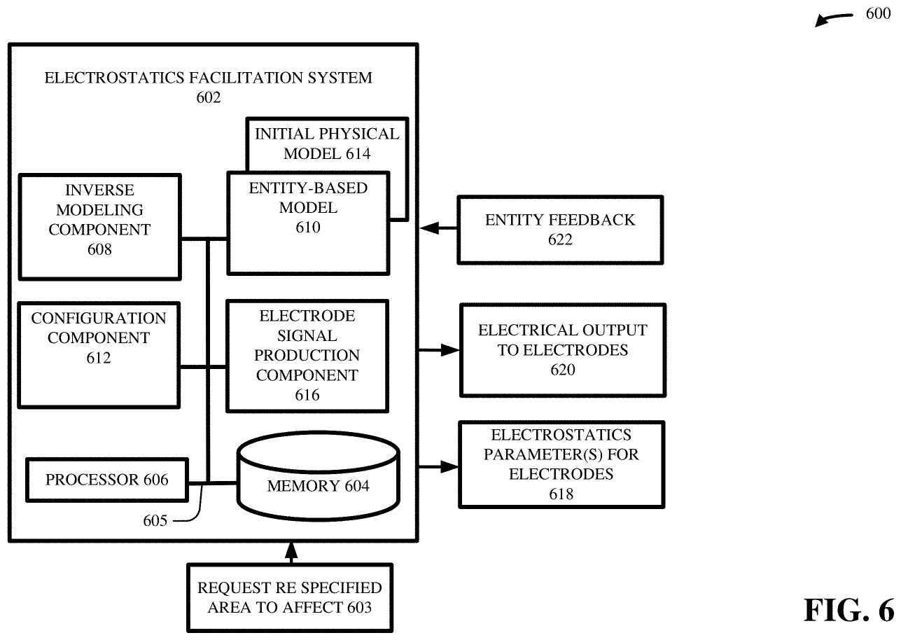

Referring now to FIG. 6, a non-limiting system 600 is illustrated for further reference. The non-limiting system 600 is substantially similar to the non-limiting system 200. Repetitive description of like elements and/or processes employed in respective embodiments is omitted for sake of brevity. Description relative to an embodiment of FIG. 2 can be applicable to an embodiment of FIG. 6. Likewise, description relative to an embodiment of FIG. 6 can be applicable to an embodiment of FIG. 2. Different from the non-limiting system 200, it will be appreciated that the non-limiting system 600 can be targeted to neuromodulation therapy of a spine of a subject entity, such as a living human entity.

For example, it will be appreciated that the request 603 can be particularly of a specified area to affect and/or of a specified motor symptom or function to affect. An electrical output 620 can be determined and provided particularly for one or more electrodes. Likewise electrostatics parameters 618 also can be determined and provided particularly for one or more electrodes. The production component 616 can be an electrode signal production component 616. For example, the electrical output 620 can be applied to the electrodes 406 at the spine 404 of FIGS. 4A and 4B, in one or more embodiments.

In summary, one or more non-limiting systems 100, 200 and/or 600, computer-implemented methods and/or computer program products to facilitate a process to produce a specified electrical output are provided. A non-limiting system 100, 200 and/or 600 can comprise a memory 104, 204, 604 that can store computer executable components and a processor 106, 206, 606 that can execute the computer executable components stored in the memory 104, 204, 604. The computer executable components can comprise an inverse modeling component 108, 208, 608 that can generate an entity-based model 110, 210, 610 and that can determine updated one or more electrostatics parameters 118, 218, 618 based on feedback 122, 222, 622 relative to application of an electrical stimulation therapy employing an initial one or more electrostatics parameters 118, 218, 618 on the entity. In one or more embodiments, a configuration component 212, 612 can determine the initial one or more electrostatics parameters 218, 618 based on an initial physical-based model 214, 614 based on an ideal entity, the configuration component 212, 612 can generate an initial physical-based model 214, 614 based on an ideal entity, and/or the inverse modeling component 108, 208, 608 can update the initial model 214, 614 to generate the entity-based model 110, 210, 610.

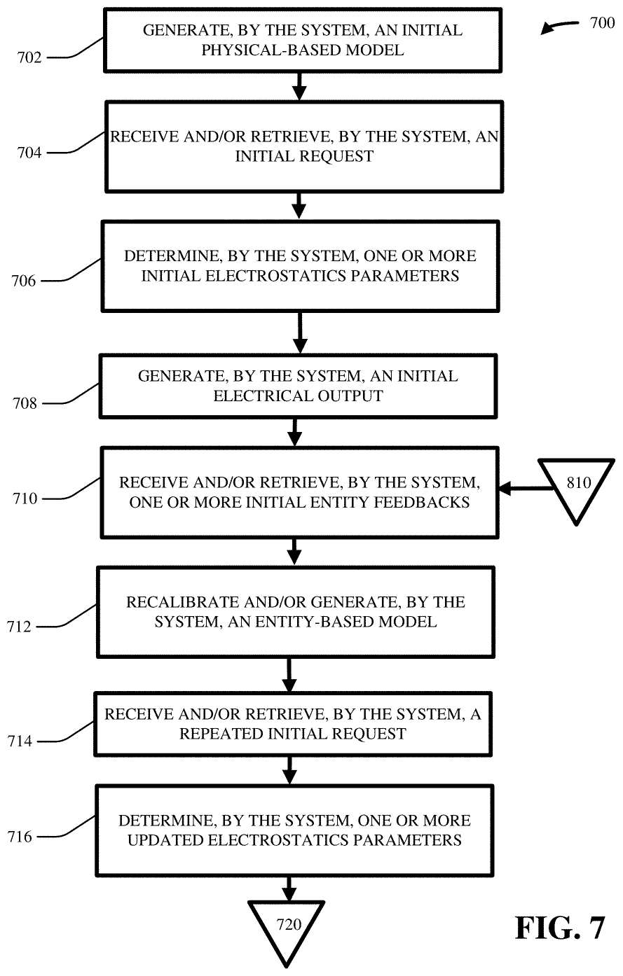

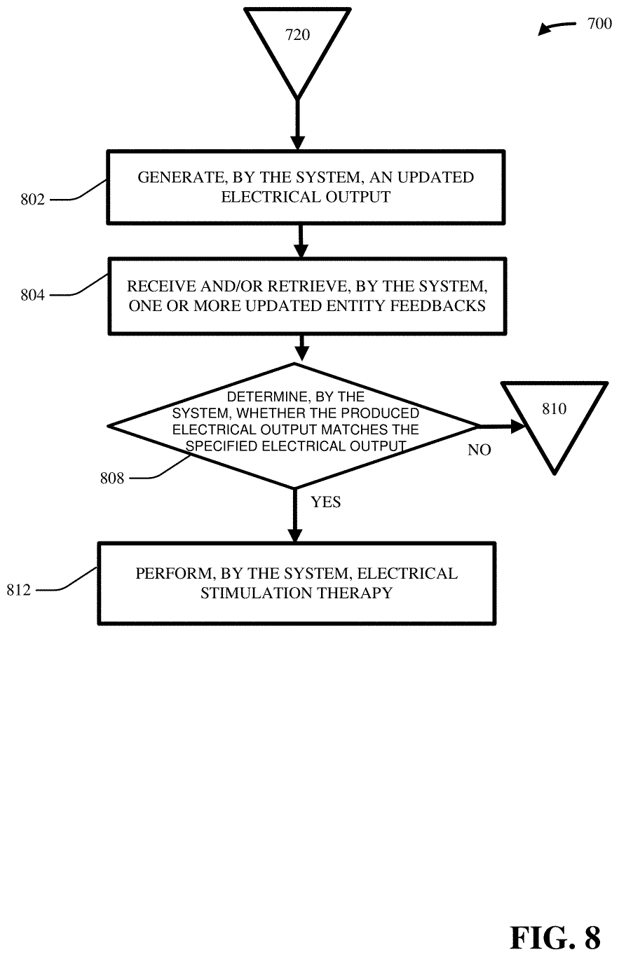

Turning now to FIGS. 7 and 8, these figures together illustrate a flow diagram of an example, non-limiting computer-implemented method 700 that can facilitate a process for producing an electrical output, in accordance with one or more embodiments described herein with respect to the non-limiting system 200. It will be appreciated that while the computer-implemented method 700 is described relative to the non-limiting system 200, the computer-implemented method 700 can be applicable also to the non-limiting system 100 and/or the non-limiting system 600. Repetitive description of like elements and/or processes employed in respective embodiments is omitted for sake of brevity.

Looking first to 702 at FIG. 7, the computer-implemented method 700 can comprise generating, by the system (e.g., configuration component 212), an initial physical-based model (e.g., physical-based model 214).

At 704, the computer-implemented method 700 can comprise receiving and/or retrieving, by the system (e.g., inverse modeling component 208), an initial request (e.g., request 203).

At 706, the computer-implemented method 700 can comprise determining, by the system (e.g., inverse modeling component 208), one or more initial electrostatics parameters (e.g., initial electrostatics parameters 218).

At 708, the computer-implemented method 700 can comprise generating, by the system (e.g., production component 216) an initial electrical output (e.g., electrical output 220).

At 710, the computer-implemented method 700 can comprise receiving and/or retrieving, by the system (e.g., inverse modeling component 208), one or more initial entity feedbacks (e.g., entity feedback 222).

At 712, the computer-implemented method 700 can comprise recalibrating and/or generating, by the system (e.g., inverse modeling component 208), an entity-based model (e.g., entity-based model 210).

At 714, the computer-implemented method 700 can comprise receiving and/or retrieving, by the system (e.g., inverse modeling component 208), an updated request (e.g., a repeated initial request 203).

At 716, the computer-implemented method 700 can comprise determining, by the system (e.g., inverse modeling component 208) one or more updated electrostatics parameters (e.g., electrostatics parameters 218).

Next, FIG. 8 illustrates a continuation of the method 700 partially illustrated at the flow diagram of FIG. 7. At FIG. 8, the method 700 of FIG. 7 is continued, represented by the continuation triangle 720 illustrated at each of FIGS. 7 and 8. Repetitive description of like elements and/or processes employed in respective embodiments is omitted for sake of brevity.

At 802, the computer-implemented method 700 can comprise generating, by the system (e.g., production component 216) an updated electrical output (e.g., electrical output 220).

At 804, the computer-implemented method 700 can comprise receiving and/or retrieving, by the system (e.g., inverse modeling component 208), one or more updated entity feedbacks (e.g., entity feedback 222).

At 808, the computer-implemented method 700 can comprise determining, by the system (e.g., inverse modeling component 208), whether the produced electrical output (e.g., electrical output 220) matches the specified electrical output (e.g., comprised by the request 203). Where the answer is no, the computer-implemented method 700 can proceed back to the recalibrating and/or generating block 712 via continuation triangle 810.

The inverse modeling component 208 can make the determination and/or the determination to proceed with one or more iterations can be made by an administrating entity. Where the inverse modeling component 208 performs the process, the inverse modeling component 208 can compare feedback, such as data, regarding the actual electrical output to determine whether the actual electrical output matches the output that was to be produced by the produced electrical output. Where there are one or more differences and/or discrepancies, the inverse modeling component 208 can determine that the entity-based model 210 should be recalibrated and or re-generated.

It will be appreciated that one or more thresholds can be applied to such determination by the inverse modeling component 208. The thresholds can be applied to one or more parameters and/or variables of the electrical outputs. Where a threshold is not met, the determination can be made to proceed with additional one or more iterations.

Where the answer is yes, at 812, the computer-implemented method 700 can comprise performing, by the system (e.g., production component 216), electrical stimulation therapy, such as at and/or on the subject entity.

For simplicity of explanation, the computer-implemented methodologies provided herein are depicted and/or described as a series of acts. It is to be understood and appreciated that the subject innovation is not limited by the acts illustrated and/or by the order of acts, for example acts can occur in one or more orders and/or concurrently, and with other acts not presented and described herein. Furthermore, not all illustrated acts can be utilized to implement the computer-implemented methodologies in accordance with the described subject matter. In addition, those skilled in the art will understand and appreciate that the computer-implemented methodologies could alternatively be represented as a series of interrelated states via a state diagram or events. Additionally, it should be further appreciated that the computer-implemented methodologies described hereinafter and throughout this specification are capable of being stored on an article of manufacture to facilitate transporting and transferring the computer-implemented methodologies to computers. The term article of manufacture, as used herein, is intended to encompass a computer program accessible from any computer-readable device or storage media.

In summary, the one or more embodiments described herein can enable improved accuracy electrical output location, speed of determination of electrostatics parameters, a shortened calibration session for an entity receiving the electrical output and/or a customized physical-based model for use in performing an electrical output on an entity. This overall improved performance can be achieved via recalibration of an initial physical-based model through employment of inverse modeling. That is, via employment of one or more iterations of entity-based feedback, one or more iterations of the recalibration can be facilitated. As compared to existing technologies that can provide an electrical output at an entity relative to a physical-based model not closely matching the entity, the one or more embodiments described herein can provide a specified electrical output and also generate an accurate entity-based physical model. Likewise, as compared to existing technologies that provide an ultimate electrical output through iterative and/or human-directed changes to electrostatics parameters, the one or more embodiments herein can provide an ultimate electrical output via one more automatic and computer-implemented updates to electrostatics parameters.

In view of the one or more embodiments, a practical application of the electrostatics facilitation systems described herein is that future applications of electrical output can be more easily, efficiently and/or quickly facilitated. Drift and/or migration of electrode placement at an entity can be more easily, efficiently and/or quickly determined. Uncertainties regarding bone, muscle and/or other tissue properties can be accounted for. Certainly, as one real-world result, unnecessary calibration sessions for different specified electrical outputs at the same entity can be avoided and/or reduced. As another real-world result, a better-targeted electrical output can be applied to an entity, such as a physical entity. In one or more cases, better targeting can provide better and/or improved stimulation of neurons, stimulation of muscle fibers, physical balance, physical stability, motion and/or other real-world results. Further, at each iteration of model generation, the generated model can be more accurate relative to the subject entity than the previous model. This inherently can allow for less iterative changes to the models, and thus respectively shorter successive iterations.

Moreover, via inverse modeling, one or more electrostatics parameters for one or more additional electrical outputs can be defaultly facilitated in view of the inverse modeling-directed recalibration and generation of an entity-based physical model. Indeed, where existing technologies focus only on iteratively determining electrostatics parameters without full comprehension of electrode location, the one or more embodiments described herein enable a better understanding of electrode location at the entity to better facilitate both the specified electrical output and one or more additional electrical outputs. That is, an effect of employing the electrostatics facilitation systems described herein is scalable electrostatics parameter determinations. This can reduce employment of processing power, memory and time of a system employed for determining and using one or more electrostatics parameters relative to an entity.

The systems and/or devices have been (and/or will be further) described herein with respect to interaction between one or more components. It should be appreciated that such systems and/or components can include those components or sub-components specified therein, one or more of the specified components and/or sub-components, and/or additional components. Sub-components can be implemented as components communicatively coupled to other components rather than included within parent components. One or more components and/or sub-components can be combined into a single component providing aggregate functionality. The components can interact with one or more other components not specifically described herein for the sake of brevity, but known by those of skill in the art.

It is to be appreciated that one or more embodiments described herein are inherently and/or inextricably tied to computer technology and cannot be implemented outside of a computing environment. For example, one or more processes performed by one or more embodiments described herein can more efficiently, and even more feasibly, provide program and/or program instruction execution as compared to existing systems and/or techniques. Systems, computer-implemented methods and/or computer program products facilitating performance of these processes are of great utility in the field of electrostatics therapy performance, such as neuromodulation therapy execution and cannot be equally practicably implemented in a sensible way outside of a computing environment.

It also is to be appreciated that one or more embodiments described herein can employ hardware and/or software to solve problems that are highly technical, that are not abstract, and that cannot be performed as a set of mental acts by a human. For example, a human, or even thousands of humans, cannot efficiently, accurately and/or effectively generate an accurate and recalibrated physical-based model in the time that one or more embodiments described herein can facilitate this process. And, neither can the human mind nor a human with pen and paper electronically generate an accurate and recalibrated physical-based model as conducted by one or more embodiments described herein.

In one or more embodiments, one or more of the processes described herein can be performed by one or more specialized computers (e.g., a specialized processing unit, a specialized classical computer, a specialized quantum computer, a specialized hybrid classical/quantum system and/or another type of specialized computer) to execute defined tasks related to the one or more technologies describe above. One or more embodiments described herein and/or components thereof can be employed to solve new problems that arise through advancements in technologies mentioned above, employment of quantum computing systems, cloud computing systems, computer architecture and/or another technology.

One or more embodiments described herein can be fully operational towards performing one or more other functions (e.g., fully powered on, fully executed and/or another function) while also performing the one or more operations described herein.

Turning next to FIGS. 9-11, a detailed description is provided of additional context for the one or more embodiments described herein at FIGS. 1-8.

FIG. 9 and the following discussion are intended to provide a brief, general description of a suitable operating environment 900 in which one or more embodiments described herein at FIGS. 1-8 can be implemented. For example, one or more components and/or other aspects of embodiments described herein can be implemented in or be associated with, such as accessible via, the operating environment 900. Further, while one or more embodiments have been described above in the general context of computer-executable instructions that can run on one or more computers, those skilled in the art will recognize that one or more embodiments also can be implemented in combination with other program modules and/or as a combination of hardware and software.

Generally, program modules include routines, programs, components, data structures and/or the like, that perform particular tasks and/or implement particular abstract data types. Moreover, those skilled in the art will appreciate that the inventive methods can be practiced with other computer system configurations, including single-processor or multiprocessor computer systems, minicomputers, mainframe computers, Internet of Things (IoT) devices, distributed computing systems, as well as personal computers, hand-held computing devices, microprocessor-based or programmable consumer electronics, and/or the like, each of which can be operatively coupled to one or more associated devices.

Computing devices typically include a variety of media, which can include computer-readable storage media, machine-readable storage media and/or communications media, which two terms are used herein differently from one another as follows. Computer-readable storage media or machine-readable storage media can be any available storage media that can be accessed by the computer and includes both volatile and nonvolatile media, removable and non-removable media. By way of example, but not limitation, computer-readable storage media and/or machine-readable storage media can be implemented in connection with any method or technology for storage of information such as computer-readable and/or machine-readable instructions, program modules, structured data and/or unstructured data.

Computer-readable storage media can include, but are not limited to, random access memory (RAM), read only memory (ROM), electrically erasable programmable read only memory (EEPROM), flash memory or other memory technology, compact disk read only memory (CD ROM), digital versatile disk (DVD), Blu-ray disc (BD) and/or other optical disk storage, magnetic cassettes, magnetic tape, magnetic disk storage and/or other magnetic storage devices, solid state drives or other solid state storage devices and/or other tangible and/or non-transitory media which can be used to store specified information. In this regard, the terms “tangible” or “non-transitory” herein as applied to storage, memory or computer-readable media, are to be understood to exclude only propagating transitory signals per se as modifiers and do not relinquish rights to all standard storage, memory and/or computer-readable media that are not only propagating transitory signals per se.

Computer-readable storage media can be accessed by one or more local or remote computing devices, e.g., via access requests, queries and/or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, but not limitation, communication media can include wired media, such as a wired network, direct-wired connection and/or wireless media such as acoustic, RF, infrared and/or other wireless media.

With reference again to FIG. 9, the example operating environment 900 for implementing one or more embodiments of the aspects described herein can include a computer 902, the computer 902 including a processing unit 906, a system memory 904 and/or a system bus 908. It will be appreciated that one or more aspects of the system memory 904 or processing unit 906 can be applied to memories such as 104, 204 and/or 604 and/or to processors such as 106, 206 and/or 606, respectively of the non-limiting systems 100, 200 and/or 600. It also will be appreciated that the system memory 904 can be implemented in combination with and/or alternatively to memories such as 104, 204 and/or 604. Likewise, it also will be appreciated that the processing unit 906 can be implemented in combination with and/or alternatively to processors such as 106, 206 and/or 606.

Memory 904 can store one or more computer and/or machine readable, writable and/or executable components and/or instructions that, when executed by processing unit 906 (e.g., a classical processor, a quantum processor and/or like processor), can facilitate performance of operations defined by the executable component(s) and/or instruction(s). For example, memory 904 can store computer and/or machine readable, writable and/or executable components and/or instructions that, when executed by processing unit 906, can facilitate execution of the one or more functions described herein relating to non-limiting systems 100, 200 and/or 600 and/or electrostatics facilitation systems such as 102, 202 and/or 602, as described herein with or without reference to the one or more figures of the one or more embodiments.

Memory 904 can comprise volatile memory (e.g., random access memory (RAM), static RAM (SRAM), dynamic RAM (DRAM) and/or the like) and/or non-volatile memory (e.g., read only memory (ROM), programmable ROM (PROM), electrically programmable ROM (EPROM), electrically erasable programmable ROM (EEPROM) and/or the like) that can employ one or more memory architectures.

Processing unit 906 can comprise one or more types of processors and/or electronic circuitry (e.g., a classical processor, a quantum processor and/or like processor) that can implement one or more computer and/or machine readable, writable and/or executable components and/or instructions that can be stored at memory 904. For example, processing unit 906 can perform one or more operations that can be specified by computer and/or machine readable, writable and/or executable components and/or instructions including, but not limited to, logic, control, input/output (I/O), arithmetic and/or the like. In one or more embodiments, processing unit 906 can be any of one or more commercially available processors. In one or more embodiments, processing unit 906 can comprise one or more central processing unit, multi-core processor, microprocessor, dual microprocessors, microcontroller, System on a Chip (SOC), array processor, vector processor, quantum processor and/or another type of processor. The examples of processing unit 906 can be employed to implement one or more embodiments described herein.

The system bus 908 can couple system components including, but not limited to, the system memory 904 to the processing unit 906. The system bus 908 can comprise one or more types of bus structure that can further interconnect to a memory bus (with or without a memory controller), a peripheral bus and/or a local bus using one or more of a variety of commercially available bus architectures. The system memory 904 can include ROM 910 and/or RAM 912. A basic input/output system (BIOS) can be stored in a non-volatile memory such as ROM, erasable programmable read only memory (EPROM) and/or EEPROM, which BIOS contains the basic routines that help to transfer information among elements within the computer 902, such as during startup. The RAM 912 can include a high-speed RAM, such as static RAM for caching data.

The computer 902 can include an internal hard disk drive (HDD) 914 (e.g., EIDE, SATA), one or more external storage devices 916 (e.g., a magnetic floppy disk drive (FDD), a memory stick or flash drive reader, a memory card reader and/or the like) and/or a drive 920, e.g., such as a solid state drive or an optical disk drive, which can read or write from a disk 922, such as a CD-ROM disc, a DVD, a BD and/or the like. Additionally and/or alternatively, where a solid state drive is involved, disk 922 could not be included, unless separate. While the internal HDD 914 is illustrated as located within the computer 902, the internal HDD 914 can also be configured for external use in a suitable chassis (not shown). Additionally, while not shown in operating environment 900, a solid state drive (SSD) can be used in addition to, or in place of, an HDD 914. The HDD 914, external storage device(s) 916 and drive 920 can be connected to the system bus 908 by an HDD interface 924, an external storage interface 926 and a drive interface 928, respectively. The HDD interface 924 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 902, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, can also be used in the example operating environment, and/or that any such storage media can contain computer-executable instructions for performing the methods described herein.

A number of program modules can be stored in the drives and RAM 912, including an operating system 930, one or more applications 932, other program modules 934 and/or program data 936. All or portions of the operating system, applications, modules and/or data can also be cached in the RAM 912. The systems and/or methods described herein can be implemented utilizing one or more commercially available operating systems and/or combinations of operating systems.

Computer 902 can optionally comprise emulation technologies. For example, a hypervisor (not shown) or other intermediary can emulate a hardware environment for operating system 930, and the emulated hardware can optionally be different from the hardware illustrated in FIG. 9. In a related embodiment, operating system 930 can comprise one virtual machine (VM) of multiple VMs hosted at computer 902. Furthermore, operating system 930 can provide runtime environments, such as the JAVA runtime environment or the .NET framework, for applications 932. Runtime environments are consistent execution environments that can allow applications 932 to run on any operating system that includes the runtime environment. Similarly, operating system 930 can support containers, and applications 932 can be in the form of containers, which are lightweight, standalone, executable packages of software that include, e.g., code, runtime, system tools, system libraries and/or settings for an application.

Further, computer 902 can be enabled with a security module, such as a trusted processing module (TPM). For instance, with a TPM, boot components hash next in time boot components and wait for a match of results to secured values before loading a next boot component. This process can take place at any layer in the code execution stack of computer 902, e.g., applied at application execution level and/or at operating system (OS) kernel level, thereby enabling security at any level of code execution.

An entity can enter and/or transmit commands and/or information into the computer 902 through one or more wired/wireless input devices, e.g., a keyboard 938, a touch screen 940 and/or a pointing device, such as a mouse 942. Other input devices (not shown) can include a microphone, an infrared (IR) remote control, a radio frequency (RF) remote control and/or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint and/or iris scanner, and/or the like. These and other input devices can be connected to the processing unit 906 through an input device interface 944 that can be coupled to the system bus 908, but can be connected by other interfaces, such as a parallel port, an IEEE 1394 serial port, a game port, a USB port, an IR interface, a BLUETOOTH® interface and/or the like.

A monitor 946 or other type of display device can be alternatively and/or additionally connected to the system bus 908 via an interface, such as a video adapter 948. In addition to the monitor 946, a computer typically includes other peripheral output devices (not shown), such as speakers, printers and/or the like.

The computer 902 can operate in a networked environment using logical connections via wired and/or wireless communications to one or more remote computers, such as a remote computer(s) 950. The remote computer(s) 950 can be a workstation, a server computer, a router, a personal computer, portable computer, microprocessor-based entertainment appliance, a peer device and/or other common network node, and typically includes many or all of the elements described relative to the computer 902, although, for purposes of brevity, only a memory/storage device 952 is illustrated. Additionally and/or alternatively, the computer 902 can be coupled (e.g., communicatively, electrically, operatively, optically and/or the like) to one or more external systems, sources and/or devices (e.g., classical and/or quantum computing devices, communication devices and/or like device) via a data cable (e.g., High-Definition Multimedia Interface (HDMI), recommended standard (RS) 232, Ethernet cable and/or the like).

In one or more embodiments, a network can comprise one or more wired and/or wireless networks, including, but not limited to, a cellular network, a wide area network (WAN) (e.g., the Internet), or a local area network (LAN). For example, one or more embodiments described herein can communicate with one or more external systems, sources and/or devices, for instance, computing devices (and vice versa) using virtually any specified wired or wireless technology, including but not limited to: wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Zigbee and other 802.XX wireless technologies and/or legacy telecommunication technologies, BLUETOOTH®, Session Initiation Protocol (SIP), ZIGBEE®, RF4CE protocol, WirelessHART protocol, 6LoWPAN (IPv6 over Low power Wireless Area Networks), Z-Wave, an ANT, an ultra-wideband (UWB) standard protocol and/or other proprietary and/or non-proprietary communication protocols. In a related example, one or more embodiments described herein can include hardware (e.g., a central processing unit (CPU), a transceiver, a decoder, quantum hardware, a quantum processor and/or the like), software (e.g., a set of threads, a set of processes, software in execution, quantum pulse schedule, quantum circuit, quantum gates and/or the like) and/or a combination of hardware and/or software that facilitates communicating information among one or more embodiments described herein and external systems, sources and/or devices (e.g., computing devices, communication devices and/or the like).

The logical connections depicted include wired/wireless connectivity to a local area network (LAN) 954 and/or larger networks, e.g., a wide area network (WAN) 956. LAN and WAN networking environments can be commonplace in offices and companies and can facilitate enterprise-wide computer networks, such as intranets, all of which can connect to a global communications network, e.g., the Internet.

When used in a LAN networking environment, the computer 902 can be connected to the local network 954 through a wired and/or wireless communication network interface or adapter 958. The adapter 958 can facilitate wired and/or wireless communication to the LAN 954, which can also include a wireless access point (AP) disposed thereon for communicating with the adapter 958 in a wireless mode.

When used in a WAN networking environment, the computer 902 can include a modem 960 and/or can be connected to a communications server on the WAN 956 via other means for establishing communications over the WAN 956, such as by way of the Internet. The modem 960, which can be internal and/or external and a wired and/or wireless device, can be connected to the system bus 908 via the input device interface 944. In a networked environment, program modules depicted relative to the computer 902 or portions thereof can be stored in the remote memory/storage device 952. It will be appreciated that the network connections shown are merely exemplary and one or more other means of establishing a communications link among the computers can be used.

When used in either a LAN or WAN networking environment, the computer 902 can access cloud storage systems or other network-based storage systems in addition to, and/or in place of, external storage devices 916 as described above, such as but not limited to, a network virtual machine providing one or more aspects of storage and/or processing of information. Generally, a connection between the computer 902 and a cloud storage system can be established over a LAN 954 or WAN 956 e.g., by the adapter 958 or modem 960, respectively. Upon connecting the computer 902 to an associated cloud storage system, the external storage interface 926 can, such as with the aid of the adapter 958 and/or modem 960, manage storage provided by the cloud storage system as it would other types of external storage. For instance, the external storage interface 926 can be configured to provide access to cloud storage sources as if those sources were physically connected to the computer 902.

The computer 902 can be operable to communicate with any wireless devices and/or entities operatively disposed in wireless communication, e.g., a printer, scanner, desktop and/or portable computer, portable data assistant, communications satellite, telephone and/or any piece of equipment or location associated with a wirelessly detectable tag (e.g., a kiosk, news stand, store shelf and/or the like). This can include Wireless Fidelity (Wi-Fi) and BLUETOOTH® wireless technologies. Thus, the communication can be a predefined structure as with a conventional network or simply an ad hoc communication between at least two devices.

The illustrated embodiments described herein can be practiced in distributed computing environments (e.g., cloud computing environments), such as described below with respect to FIG. 15, where certain tasks are performed by remote processing devices that are linked through a communications network. In a distributed computing environment, program modules can be located both in local and/or remote memory storage devices.

For example, one or more embodiments described herein and/or one or more components thereof can employ one or more computing resources of the cloud computing environment 1550 described below with reference to FIG. 15, and/or with reference to the one or more functional abstraction layers (e.g., quantum software and/or the like) described below with reference to FIG. 16, to execute one or more operations in accordance with one or more embodiments described herein. For example, cloud computing environment 1550 and/or one or more of the functional abstraction layers 1660, 1670, 1680 and/or 1690 can comprise one or more classical computing devices (e.g., classical computer, classical processor, virtual machine, server and/or the like), quantum hardware and/or quantum software (e.g., quantum computing device, quantum computer, quantum processor, quantum circuit simulation software, superconducting circuit and/or the like) that can be employed by one or more embodiments described herein and/or components thereof to execute one or more operations in accordance with one or more embodiments described herein. For instance, one or more embodiments described herein and/or components thereof can employ such one or more classical and/or quantum computing resources to execute one or more classical and/or quantum: mathematical function, calculation and/or equation; computing and/or processing script; algorithm; model (e.g., artificial intelligence (AI) model, machine learning (ML) model and/or like model); and/or other operation in accordance with one or more embodiments described herein.

It is to be understood that although one or more embodiments described herein include a detailed description on cloud computing, implementation of the teachings recited herein are not limited to a cloud computing environment. Rather, one or more embodiments described herein are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines and services) that can be rapidly provisioned and released with minimal management effort or interaction with a provider of the service. This cloud model can include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand There is a sense of location independence in that the consumer generally has no control or knowledge over the exact location of the provided resources but can specify location at a higher level of abstraction (e.g., country, state and/or datacenter).

Rapid elasticity: capabilities can be rapidly and elastically provisioned, in one or more cases automatically, to quickly scale out and rapidly released to quickly scale in. To the consumer, the capabilities available for provisioning can appear to be unlimited and can be purchased in any quantity at any time.

Measured service: cloud systems automatically control and optimize resource use by leveraging a metering capability at one or more levels of abstraction appropriate to the type of service (e.g., storage, processing, bandwidth and/or active user accounts). Resource usage can be monitored, controlled and/or reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage and/or individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems and/or storage, but has control over the deployed applications and possibly application hosting environment configurations.

Infrastructure as a Service (IaaS): the capability provided to the consumer is to provision processing, storage, networks and/or other fundamental computing resources where the consumer can deploy and run arbitrary software, which can include operating systems and applications. The consumer does not manage or control the underlying cloud infrastructure but has control over operating systems, storage, deployed applications and/or possibly limited control of select networking components (e.g., host firewalls).

Deployment Models are as follows:

Private cloud: the cloud infrastructure is operated solely for an organization. It can be managed by the organization or a third party and can exist on-premises or off-premises.

Community cloud: the cloud infrastructure is shared by several organizations and supports a specific community that has shared concerns (e.g., mission, security requirements, policy and/or compliance considerations). It can be managed by the organizations or a third party and can exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community or public) that remain unique entities but are bound together by standardized or proprietary technology that enables data and application portability (e.g., cloud bursting for load-balancing among clouds).

A cloud computing environment is service oriented with a focus on statelessness, low coupling, modularity and/or semantic interoperability. At the heart of cloud computing is an infrastructure that includes a network of interconnected nodes.

Moreover, the non-limiting systems 100, 300 and/or 400 and/or the example operating environment 900 can be associated with and/or be included in a data analytics system, a data processing system, a graph analytics system, a graph processing system, a big data system, a social network system, a speech recognition system, an image recognition system, a graphical modeling system, a bioinformatics system, a data compression system, an artificial intelligence system, an authentication system, a syntactic pattern recognition system, a medical system, a health monitoring system, a network system, a computer network system, a communication system, a router system, a server system, a high availability server system (e.g., a Telecom server system), a Web server system, a file server system, a data server system, a disk array system, a powered insertion board system, a cloud-based system and/or the like. In accordance therewith, non-limiting systems 100, 300 and/or 400 and/or example operating environment 900 can be employed to use hardware and/or software to solve problems that are highly technical in nature, that are not abstract and/or that cannot be performed as a set of mental acts by a human.

Referring now to details of one or more aspects illustrated at FIG. 10, the illustrative cloud computing environment 1050 is depicted. As shown, cloud computing environment 1050 includes one or more cloud computing nodes 1010 with which local computing devices used by cloud consumers, such as, for example, personal digital assistant (PDA) or cellular telephone 1054A, desktop computer 1054B, laptop computer 1054C and/or automobile computer system 1054N can communicate. Although not illustrated in FIG. 10, cloud computing nodes 1010 can further comprise a quantum platform (e.g., quantum computer, quantum hardware, quantum software and/or the like) with which local computing devices used by cloud consumers can communicate. Cloud computing nodes 1010 can communicate with one another. They can be grouped (not shown) physically or virtually, in one or more networks, such as Private, Community, Public, or Hybrid clouds as described hereinabove, or a combination thereof. This allows cloud computing environment 1050 to offer infrastructure, platforms and/or software as services for which a cloud consumer does not need to maintain resources on a local computing device. It is understood that the types of computing devices 1054A-N shown in FIG. 10 are intended to be illustrative only and that cloud computing nodes 1010 and cloud computing environment 1050 can communicate with any type of computerized device over any type of network and/or network addressable connection (e.g., using a web browser).

Referring now to details of one or more aspects illustrated at FIG. 11, a set 1100 of functional abstraction layers is shown, such as provided by cloud computing environment 1050 (FIG. 10). One or more embodiments described herein can be associated with, such as accessible via, one or more functional abstraction layers described below with reference to FIG. 11 (e.g., hardware and software layer 1160, virtualization layer 1170, management layer 1180 and/or workloads layer 1190). It should be understood in advance that the components, layers and/or functions shown in FIG. 11 are intended to be illustrative only and embodiments described herein are not limited thereto. As depicted, the following layers and/or corresponding functions are provided:

Hardware and software layer 1160 can include hardware and software components. Examples of hardware components include: mainframes 1161; RISC (Reduced Instruction Set Computer) architecture-based servers 1162; servers 1163; blade servers 1164; storage devices 1165; and/or networks and/or networking components 1166. In one or more embodiments, software components can include network application server software 1167, quantum platform routing software 1168; and/or quantum software (not illustrated in FIG. 11).

Virtualization layer 1170 can provide an abstraction layer from which the following examples of virtual entities can be provided: virtual servers 1171; virtual storage 1172; virtual networks 1173, including virtual private networks; virtual applications and/or operating systems 1174; and/or virtual clients 1175.

In one example, management layer 1180 can provide the functions described below. Resource provisioning 1181 can provide dynamic procurement of computing resources and other resources that can be utilized to perform tasks within the cloud computing environment. Metering and Pricing 1182 can provide cost tracking as resources are utilized within the cloud computing environment, and/or billing and/or invoicing for consumption of these resources. In one example, these resources can include one or more application software licenses. Security can provide identity verification for cloud consumers and/or tasks, as well as protection for data and/or other resources. User (or entity) portal 1183 can provide access to the cloud computing environment for consumers and system administrators. Service level management 1184 can provide cloud computing resource allocation and/or management such that required service levels are met. Service Level Agreement (SLA) planning and fulfillment 1185 can provide pre-arrangement for, and procurement of, cloud computing resources for which a future requirement is anticipated in accordance with an SLA.

Workloads layer 1190 can provide examples of functionality for which the cloud computing environment can be utilized. Non-limiting examples of workloads and functions which can be provided from this layer include: mapping and navigation 1191; software development and lifecycle management 1192; virtual classroom education delivery 1193; data analytics processing 1194; transaction processing 1195; and/or application transformation software 1196.

The embodiments described herein can be directed to one or more of a system, a method, an apparatus and/or a computer program product at any possible technical detail level of integration. The computer program product can include a computer readable storage medium (or media) having computer readable program instructions thereon for causing a processor to carry out aspects of the one or more embodiments described herein. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device and/or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon and/or any suitable combination of the foregoing. A computer readable storage medium, as used herein, is not to be construed as being transitory signals per se, such as radio waves and/or other freely propagating electromagnetic waves, electromagnetic waves propagating through a waveguide and/or other transmission media (e.g., light pulses passing through a fiber-optic cable), and/or electrical signals transmitted through a wire.

Computer readable program instructions described herein can be downloaded to respective computing/processing devices from a computer readable storage medium and/or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the one or more embodiments described herein can be assembler instructions, instruction-set-architecture (ISA) instructions, machine instructions, machine dependent instructions, microcode, firmware instructions, state-setting data, configuration data for integrated circuitry, and/or source code and/or object code written in any combination of one or more programming languages, including an object oriented programming language such as Smalltalk, C++ or the like, and/or procedural programming languages, such as the “C” programming language and/or similar programming languages. The computer readable program instructions can execute entirely on a computer, partly on a computer, as a stand-alone software package, partly on a computer and/or partly on a remote computer or entirely on the remote computer and/or server. In the latter scenario, the remote computer can be connected to a computer through any type of network, including a local area network (LAN) and/or a wide area network (WAN), and/or the connection can be made to an external computer (for example, through the Internet using an Internet Service Provider). In one or more embodiments, electronic circuitry including, for example, programmable logic circuitry, field-programmable gate arrays (FPGA) and/or programmable logic arrays (PLA) can execute the computer readable program instructions by utilizing state information of the computer readable program instructions to personalize the electronic circuitry, in order to perform aspects of the one or more embodiments described herein.

Aspects of the one or more embodiments described herein are described with reference to flowchart illustrations and/or block diagrams of methods, apparatus (systems), and computer program products according to one or more embodiments described herein. It will be understood that each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer and/or other programmable data processing apparatus to produce a machine, such that the instructions, which execute via the processor of the computer or other programmable data processing apparatus, can create means for implementing the functions/acts specified in the flowchart and/or block diagram block or blocks. These computer readable program instructions can also be stored in a computer readable storage medium that can direct a computer, a programmable data processing apparatus and/or other devices to function in a particular manner, such that the computer readable storage medium having instructions stored therein can comprise an article of manufacture including instructions which can implement aspects of the function/act specified in the flowchart and/or block diagram block or blocks. The computer readable program instructions can also be loaded onto a computer, other programmable data processing apparatus and/or other device to cause a series of operational acts to be performed on the computer, other programmable apparatus and/or other device to produce a computer implemented process, such that the instructions which execute on the computer, other programmable apparatus and/or other device implement the functions/acts specified in the flowchart and/or block diagram block or blocks.

The flowcharts and block diagrams in the figures illustrate the architecture, functionality and/or operation of possible implementations of systems, computer-implementable methods and/or computer program products according to one or more embodiments described herein. In this regard, each block in the flowchart or block diagrams can represent a module, segment and/or portion of instructions, which comprises one or more executable instructions for implementing the specified logical function(s). In one or more alternative implementations, the functions noted in the blocks can occur out of the order noted in the Figures. For example, two blocks shown in succession can be executed substantially concurrently, and/or the blocks can sometimes be executed in the reverse order, depending upon the functionality involved. It will also be noted that each block of the block diagrams and/or flowchart illustration, and/or combinations of blocks in the block diagrams and/or flowchart illustration, can be implemented by special purpose hardware-based systems that can perform the specified functions and/or acts and/or carry out one or more combinations of special purpose hardware and/or computer instructions.

While the subject matter has been described above in the general context of computer-executable instructions of a computer program product that runs on a computer and/or computers, those skilled in the art will recognize that the one or more embodiments herein also can be implemented in combination with one or more other program modules. Generally, program modules include routines, programs, components, data structures and/or the like that perform particular tasks and/or implement particular abstract data types. Moreover, those skilled in the art will appreciate that the inventive computer-implemented methods can be practiced with other computer system configurations, including single-processor and/or multiprocessor computer systems, mini-computing devices, mainframe computers, as well as computers, hand-held computing devices (e.g., PDA, phone), microprocessor-based or programmable consumer and/or industrial electronics and/or the like. The illustrated aspects can also be practiced in distributed computing environments in which tasks are performed by remote processing devices that are linked through a communications network. However, one or more, if not all aspects of the one or more embodiments described herein can be practiced on stand-alone computers. In a distributed computing environment, program modules can be located in both local and remote memory storage devices.

As used in this application, the terms “component,” “system,” “platform,” “interface,” and/or the like, can refer to and/or can include a computer-related entity or an entity related to an operational machine with one or more specific functionalities. The entities described herein can be either hardware, a combination of hardware and software, software, or software in execution. For example, a component can be, but is not limited to being, a process running on a processor, a processor, an object, an executable, a thread of execution, a program and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system and/or across a network such as the Internet with other systems via the signal). As another example, a component can be an apparatus with specific functionality provided by mechanical parts operated by electric or electronic circuitry, which is operated by a software and/or firmware application executed by a processor. In such a case, the processor can be internal and/or external to the apparatus and can execute at least a part of the software and/or firmware application. As yet another example, a component can be an apparatus that provides specific functionality through electronic components without mechanical parts, where the electronic components can include a processor and/or other means to execute software and/or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter described herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit and/or device comprising, but not limited to, single-core processors; single-processors with software multithread execution capability; multi-core processors; multi-core processors with software multithread execution capability; multi-core processors with hardware multithread technology; parallel platforms; and/or parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, and/or any combination thereof designed to perform the functions described herein. Further, processors can exploit nano-scale architectures such as, but not limited to, molecular and quantum-dot based transistors, switches and/or gates, in order to optimize space usage and/or to enhance performance of related equipment. A processor can be implemented as a combination of computing processing units.

Herein, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory and/or nonvolatile random access memory (RAM) (e.g., ferroelectric RAM (FeRAM). Volatile memory can include RAM, which can act as external cache memory, for example. By way of illustration and not limitation, RAM can be available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM) and/or Rambus dynamic RAM (RDRAM). Additionally, the described memory components of systems and/or computer-implemented methods described herein are intended to include, without being limited to including, these and/or other suitable types of memory.

What has been described above includes mere examples of systems and computer-implemented methods. It is, of course, not possible to describe every conceivable combination of components and/or computer-implemented methods for purposes of describing the one or more embodiments, but one of ordinary skill in the art can recognize that many further combinations and/or permutations of the one or more embodiments are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the one or more embodiments have been presented for purposes of illustration but are not intended to be exhaustive or limited to the embodiments described herein. Many modifications and variations will be apparent to those of ordinary skill in the art without departing from the scope and spirit of the described embodiments. The terminology used herein was chosen to best explain the principles of the embodiments, the practical application and/or technical improvement over technologies found in the marketplace, and/or to enable others of ordinary skill in the art to understand the embodiments described herein.

The claims and scope of the subject application, and any continuation, divisional or continuation-in-part applications claiming priority to the subject application, exclude embodiments (e.g., systems, apparatus, methodologies, computer program products and computer readable storage media) directed to implanted electrical stimulation for pain treatment and/or management.

What is claimed is:

1. A system, comprising:
a memory that stores computer executable components; and
a processor that executes the computer executable components stored in the memory, wherein the computer executable components comprise:
an inverse modeling component that generates an entity-based model and determines updated one or more electrostatics parameters based on feedback relative to application of an electrical stimulation therapy employing an initial one or more electrostatics parameters on the entity.

2. The system of claim 1, further comprising:
a configuration component that determines the initial one or more electrostatics parameters based on an initial physical-based model based on an ideal entity.

3. The system of claim 1, further comprising:
a configuration component that generates an initial physical-based model based on an ideal entity, wherein the inverse modeling component updates the initial model to generate the entity-based model.

4. The system of claim 1,
wherein the inverse modeling component performs a recalibration of an initial physical-based model to generate the entity-based model, and wherein the recalibration is performed between a determination of the initial one or more electrostatics parameters and the determination of the updated one or more electrostatics parameters.

5. The system of claim 1,
wherein the updated one or more electrostatics parameters are determined based on feedback relative to application of the electrical stimulation therapy being a neuromodulation therapy.

6. The system of claim 1, further comprising:
a production component that produces a produced electrical output to the entity by employing the updated one or more electrostatics parameters.

7. The system of claim 6,
wherein the produced electrical output matches a specified electrical output upon which the initial one or more electrostatics parameters were based.

8. The system of claim 1,
wherein the updated one or more electrostatics parameters define a supplementary electrode configuration for producing a supplementary electric field employing one or more electrodes at the entity.

9. A computer-implemented method, comprising:
generating, by a system operatively coupled to a processor, an entity-based model; and
determining, by the system, updated one or more electrostatics parameters based on feedback relative to application of an electrical stimulation therapy employing an initial one or more electrostatics parameters on the entity.

10. The computer-implemented method of claim 9, further comprising:
determining, by the system, the initial one or more electrostatics parameters based on an initial physical-based model based on an ideal entity.

11. The computer-implemented method of claim 9, further comprising:
generating, by the system, an initial physical-based model based on an ideal entity; and
updating, by the system, the initial model to generate the entity-based model.

12. The computer-implemented method of claim 9, further comprising:
performing, by the system, a recalibration of an initial physical-based model to generate the entity-based model between a determining of the initial one or more electrostatics parameters and the determining of the updated one or more electrostatics parameters.

13. The computer-implemented method of claim 9, further comprising:
determining, by the system, the updated one or more electrostatics parameters based on feedback relative to application of the electrical stimulation therapy being a neuromodulation therapy.

14. The computer-implemented method of claim 9, further comprising:
producing, by the system, a produced electrical output to the entity by employing the updated one or more electrostatics parameters, wherein the produced electrical output matches a specified electrical output upon which the initial one or more electrostatics parameters were based.

15. A computer program product facilitating a process to produce a specified electrical output, the computer program product comprising a computer readable storage medium having program instructions embodied therewith, the program instructions executable by a processor to cause the processor to:
generate, by the processor, an entity-based model; and
determine, by the processor, updated one or more electrostatics parameters based on feedback relative to application of an electrical stimulation therapy employing an initial one or more electrostatics parameters on the entity.

16. The computer program product of claim 15, wherein the program instructions are further executable by the processor to:
determine, by the processor, the initial one or more electrostatics parameters based on an initial physical-based model based on an ideal entity.

17. The computer program product of claim 15, wherein the program instructions are further executable by the processor to:
generate, by the processor, an initial physical-based model based on an ideal entity; and
update, by the processor, the initial model to generate the entity-based model.

18. The computer program product of claim 15, wherein the program instructions are further executable by the processor to:
perform, by the processor, a recalibration of an initial physical-based model to generate the entity-based model between a determining of the initial one or more electrostatics parameters and the determining of the updated one or more electrostatics parameters.

19. The computer program product of claim 15, wherein the program instructions are further executable by the processor to:
determine, by the processor, the updated one or more electrostatics parameters based on feedback relative to application of the electrical stimulation therapy being a neuromodulation therapy.

20. The computer program product of claim 15, wherein the program instructions are further executable by the processor to:
produce, by the processor, a produced electrical output to the entity by employing the updated one or more electrostatics parameters, wherein the produced electrical output matches a specified electrical output upon which the initial one or more electrostatics parameters were based.
